(19) United States
(12) Patent Application Publication
(10) Pub. No.: US 2026/0085951 A1
(43) Pub. Date: Mar. 26, 2026

(54) SENSOR GUARD FOR PROTECTING GROUNDWATER MONITORING SENSOR

(71) Applicants: ENIN CO., LTD., Incheon (KR); Korea Radioactive Waste Agency, Gyeongju-si (KR)

(72) Inventors: Whun LEE, Gwangmyeong-si (KR); Geun Taek LIM, Incheon (KR); Geon Ho LEE, Bucheon-si (KR); Soon Il OK, Busan (KR)

(73) Assignees: ENIN CO., LTD., Incheon (KR); Korea Radioactive Waste Agency, Gyeongju-si (KR)

(21) Appl. No.: 19/339,884

(22) Filed: Sep. 25, 2025

(30) Foreign Application Priority Data
Sep. 25, 2024 (KR) 10-2024-0129511

(51) Int. Cl. G01D 11/24; G01D 11/30; G01N 33/18

(52) U.S. Cl. CPC G01D 11/245; G01D 11/30; G01N 33/1886

(57) ABSTRACT

According to an embodiment of the present disclosure, a sensor guard installable on various types of groundwater monitoring sensors inserted into a groundwater well may include at least one sensor fixing module fixed to the groundwater monitoring sensor in one direction of the groundwater monitoring sensor, and an expansion module connected to the sensor fixing module to expand in an outward direction of the groundwater monitoring sensor according to an operation of the sensor fixing module, wherein an external force applied to the sensor guard is buffered by the expansion module.

### CROSS-REFERENCE TO RELATED APPLICATION

This application claims priority to and the benefit of Korean Patent Application No. 2024-0129511, filed on Sep. 25, 2024, the disclosure of which is incorporated herein by reference in its entirety.

### BACKGROUND

### 1. Field of the Invention

The technical idea of the present disclosure relates to a sensor guard, and more particularly, to a sensor guard for protecting a groundwater monitoring sensor.

### 2. Discussion of Related Art

A groundwater well may refer to a hole or structure installed downward from a ground surface to extract or monitor groundwater. A groundwater well is a system designed to reach an aquifer to obtain or monitor groundwater by pumping the groundwater present in the aquifer or allowing the groundwater to naturally flow out thereof. Groundwater monitoring sensors are installed in groundwater wells to monitor groundwater. These groundwater monitoring sensors are thrown and installed at a depth of tens of meters underground or are installed directly by divers to prevent damage due to throwing.

Damage to sensors, when the sensors are inserted into a groundwater well, may occur due to various causes. Due to a narrow space and a rough surface inside a well, sensors may be damaged by being scratched or bumped during installation, and foreign materials or sediments that may be present inside the well may be attached to sensitive parts of the sensors to cause functional degradation.

In addition, when excessive pressure or impact is applied during a sensor insertion process, there is a risk of damage to internal electronic components. Accordingly, when proper protective devices or guides are not used during a sensor installation process, sensors may be damaged during installation to malfunction.

Conventional protective devices for protecting sensors may protect only storage bottles for protecting sensors or specific types, specific brands, or specific models of sensors during storage, thereby lacking versatility. In addition, conventional protective devices have a large size and thus have a disadvantage of being difficult to use in narrow wells into which sensors are inserted in a way that groundwater monitoring sensors are inserted.

### SUMMARY OF THE INVENTION

The present disclosure is directed to providing a sensor guard capable of being inserted through a narrow groundwater well together with a groundwater monitoring sensor.

According to an aspect of the present invention, there is provided a sensor guard installable on various types of groundwater monitoring sensors inserted into a groundwater well, which includes at least one sensor fixing module fixed to the groundwater monitoring sensor in one direction of the groundwater monitoring sensor, and an expansion module connected to the sensor fixing module to expand in an outward direction of the groundwater monitoring sensor according to an operation of the sensor fixing module, wherein an external force applied to the sensor guard is buffered by the expansion module.

The sensor fixing module may be provided as a plurality of sensor fixing modules, and the expansion module may be connected between the plurality of sensor fixing modules.

Each of the sensor fixing modules may include a plurality of horizontal parts surrounding the groundwater monitoring sensor, and the horizontal parts may be connected through a wire by which an interval between the horizontal parts is adjusted.

In the expansion module, an elastic part bendable in an inward or outward direction of the groundwater monitoring sensor may be connected to each of the horizontal parts, and a degree of expansion of the elastic part outward from the groundwater monitoring sensor may be determined according to an interval between the plurality of sensor fixing modules.

Each of the sensor fixing modules may include a dial on which the wire is wound, and when the dial rotates in a first direction, the wire may be wound on the dial so that the interval between the horizontal parts decreases.

The dial may be locked not to rotate in a second direction opposite to the first direction, and when the dial is pushed toward the groundwater monitoring sensor, the dial may be unlocked to allow rotation in the second direction.

When the dial rotates in the second direction, the interval between the horizontal parts may increase.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIGS. 1 and 2 are views illustrating types of groundwater monitoring sensors installed through groundwater wells according to one embodiment;

FIG. 3 is a view illustrating components of a sensor guard according to an embodiment of the present disclosure;

FIG. 4 is a view illustrating a state in which a sensor fixing module is connected to an expansion module according to one embodiment;

FIG. 5 is a view illustrating an embodiment in which an interval between horizontal parts is adjusted by rotating a dial according to one embodiment;

FIG. 6 is a view illustrating a state in which the sensor fixing module is in close contact with a groundwater monitoring sensor according to one embodiment;

FIG. 7 is a view illustrating an embodiment in which the expansion module is expanded by operating the sensor fixing module according to one embodiment;

FIG. 8 is a view illustrating the dial according to one embodiment; and

FIGS. 9A-9B show views illustrating an embodiment in which the sensor guard is installed on each of groundwater monitoring sensors with a plurality of sizes according to one embodiment.

### DETAILED DESCRIPTION OF EXEMPLARY EMBODIMENTS

Throughout the present disclosure, the same reference numerals refer to the same components. The present disclosure does not describe all components of embodiments, and general contents or duplicated contents between the embodiments in the technical field to which the present disclosure pertains will be omitted. The terms “unit, module, member, and block” used herein may be implemented in software or hardware, and according to embodiments, the plurality of “units, modules, members, and blocks” may be implemented in one component or one “unit, module, member, and block” may include a plurality of components.

Throughout the specification, when it is described that a first component is “connected” to a second component, this includes not only a case in which the first component is directly connected to the second component but also a case in which the first component is indirectly connected to the second component, and the indirect connection includes connection through a wireless communication network.

Further, when a part “includes” a component, this means that a third component is not excluded but may be further included unless otherwise stated.

Throughout the specification, when a first member is located “on” a second member, this case includes not only a case in which the first member is in contact with the second member but also a case in which a third member is present between the two members.

Terms such as first and second are used to distinguish one component from another component, and components are not limited by the above-described terms.

Singular expressions include plural expressions unless clearly otherwise indicated in the context.

In each operation, an identification code is used for convenience of description and does not describe a sequence of the operations, and an operation may be performed in a different order from a specified order unless the context clearly states a specific order.

Hereinafter, the operating principles and embodiments of the present disclosure will be described with reference to the accompanying drawings.

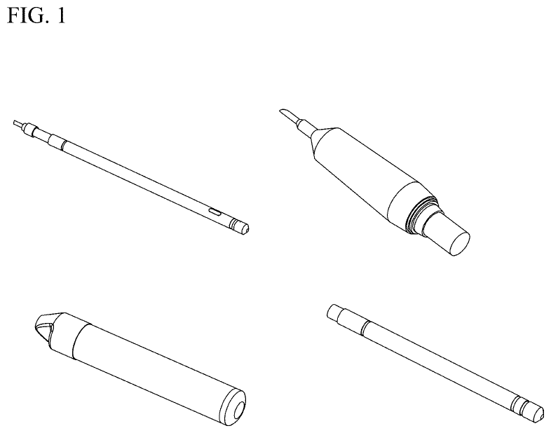

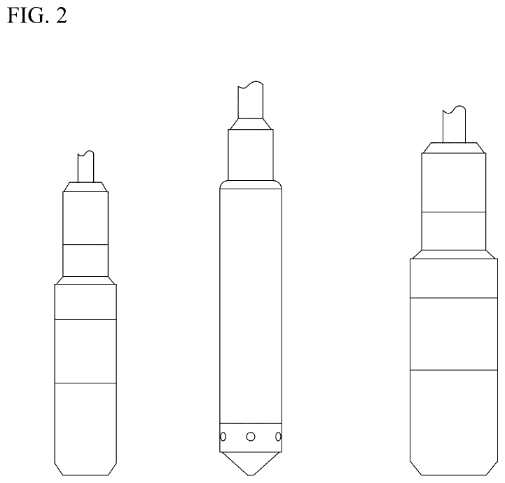

FIGS. 1 and 2 are views illustrating types of groundwater monitoring sensors installed through groundwater wells according to one embodiment.

Referring to FIG. 1, the groundwater monitoring sensor may be formed in a cylindrical shape to be efficiently inserted into a narrow space such as the inside of a groundwater well. A cylindrical sensor may be designed to minimize a space required inside a well to reduce interference with other equipment or structures and have high strength to withstand external impacts or pressures. In addition, a cylindrical structure facilitates maintenance and replacement work, thereby simplifying work of lifting the groundwater monitoring sensor from the well for inspection or replacement.

The groundwater monitoring sensor may include a water level sensor, a water quality sensor, water temperature sensor, a conductivity sensor, a multi-parameter sensor, a water leak detection sensor, and an optical sensor. The water level sensor may measure a water level of groundwater to monitor a change in water level and may operate in a pressure or ultrasonic manner. The water quality sensor may measure chemical and physical properties of groundwater to monitor a water quality state and may operates as a pH sensor, an electrical conductivity sensor, an oxidation-reduction potential (ORP) sensor, a dissolved oxygen sensor, a turbidity sensor, or an ion-selective electrode sensor. In addition, the water temperature sensor may measure a temperature of groundwater to monitor a temperature change, and the conductivity sensor may measure the electrical conductivity of groundwater to indirectly measure a concentration of dissolved ions.

Referring to FIG. 2, these various types of groundwater monitoring sensors may be provided in a cylindrical shape while having different heights and diameters. These groundwater monitoring sensors may be installed at different depths according to a position of an aquifer. Alternatively, a size of a selected groundwater monitoring sensor may vary according to a water level of groundwater level or a diameter of a well.

For example, a groundwater monitoring sensor with a small diameter may be used in a small well to secure a sufficient space therein to prevent the sensor from colliding with a well wall. On the other hand, a sensor with a larger diameter may be used in a large-diameter well to perform various monitoring functions.

According to FIGS. 1 and 2, the groundwater monitoring sensors may have different diameters and heights according to their purpose and installation positions. There is a growing need for a sensor guard that may be universally used in such groundwater monitoring sensors.

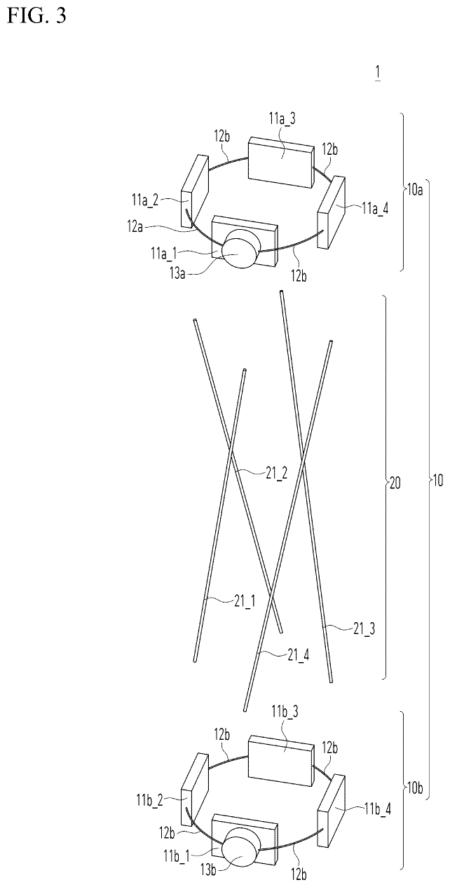

FIG. 3 is a view illustrating components of a sensor guard 1 according to an embodiment of the present disclosure.

Referring to FIG. 3, the sensor guard 1 of the present disclosure may include a sensor fixing module 10 that may be installed on various types of groundwater monitoring sensors and an expansion module 20 that may buffer an external force acting on the groundwater monitoring sensor.

The sensor fixing module 10 may include parts that are vertically separated. For example, the sensor fixing module may include a first sensor fixing module 10a and a second sensor fixing module 10b which have the same shape. The first sensor fixing module 10a may be installed at an upper portion of the groundwater monitoring sensor, and the second sensor fixing module 10b may be installed at a lower portion of the groundwater monitoring sensor.

The expansion module 20 may be disposed between the first sensor fixing module 10a and the second sensor fixing module 10b. For example, the expansion module 20 may include an elastic part 21 that is bendable an inward or outward direction of the groundwater monitoring sensor. The elastic part 21 may connect the first sensor fixing module 10a and the second sensor fixing module 10b.

According to one embodiment, each of the first sensor fixing module 10a and the second sensor fixing module 10b may include a plurality of horizontal parts 11. Horizontal parts 11a of the first sensor fixing module 10a may be connected through a first wire 12a, and horizontal parts 11b of the second sensor fixing module 10b may be connected through a second wire 12b. When the sensor guard 1 is installed on the groundwater monitoring sensor, the horizontal parts 11 may be in close contact with the groundwater monitoring sensor to be fixed to the groundwater monitoring sensor. To this end, at least a portion of each of the horizontal parts 11 may be made of an elastic material to be tightly in close contact with the groundwater monitoring sensor.

The horizontal parts 11 of each sensor fixing module 10 may be connected through a wire 12. For example, the horizontal part 11 may have a small hollow through which the wire 12 may pass, and the wire 12 may pass through the hollow to connect the horizontal parts 11.

The wire 12 connecting respective horizontal parts 11 may be tied to a dial 13. The dial 13 may be mounted on any one of the plurality of horizontal parts 11, and when the dial 13 rotates in a first direction, the wire 12 may be wound on the dial 13. When the wire 12 is wound on the dial 13, an interval between the horizontal parts 11 may decrease, and the sensor guard 1 may accommodate a groundwater monitoring sensor with a smaller diameter.

In this case, the dial 13 may be locked not to rotate in a second direction which is a direction opposite to the first direction. That is, the dial 13 may be fixed such that the wire 12 is not unwound after the wire 12 is wound, and accordingly, the interval between the horizontal parts 11 may be decreased to fit a diameter of the groundwater monitoring sensor and then fixed to increase. By locking the horizontal parts 11 in close contact with the groundwater monitoring sensor, the sensor guard 1 and the groundwater monitoring sensor may be fixed not to be separated from each other.

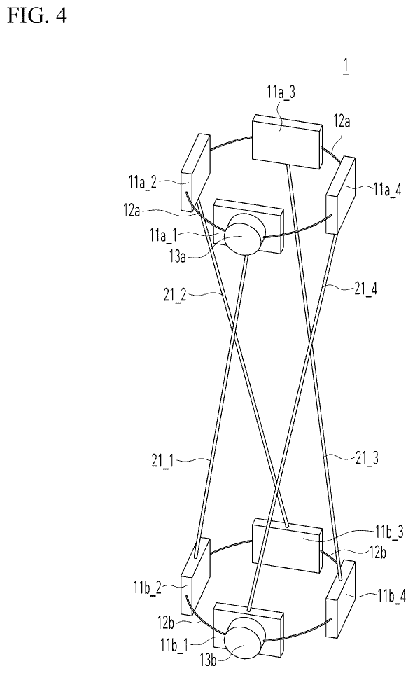

FIG. 4 is a view illustrating a state in which the sensor fixing module is connected to the expansion module 20 according to one embodiment.

Referring to FIG. 4, the elastic part 21 of the expansion module 20 may be made of a material that is bendable in the inward or outward direction of the groundwater monitoring sensor and may be connected to each horizontal part 11 of a pair of sensor fixing modules 10. For example, the elastic part 21 may be made of any material such as stainless steel, polyurethane, silicone rubber, or polyvinyl chloride (PVC) and may be formed in the form of a cable that is connected between a pair of sensor fixing modules 10a and 10b to buffer an external force.

According to one embodiment, the number of the elastic parts 21 of the expansion module 20 may be the same as the number of the horizontal parts 11, and the elastic part 21 may be connected to each horizontal part 11a of the first sensor fixing module 10a and each horizontal part 11b of the second sensor fixing module 10b. It is described in the embodiment of FIG. 3 that four horizontal parts 11 are connected to four elastic parts 21, but the numbers of the horizontal parts 11 and the elastic parts 21 of the embodiment of the present disclosure are not limited thereto.

For example, a first elastic part 21_1 of the expansion module 20 may be connected to a first horizontal part 11a_1 of the first sensor fixing module 10a and a second horizontal part 11b_2 of the second sensor fixing module 10b, and a second elastic part 21_2 may be connected to a second horizontal part 11a_2 of the first sensor fixing module 10a and a third horizontal part 11b_3 of the second sensor fixing module 10b. A third elastic part 21_3 of the expansion module 20 may be connected to a third horizontal part 11a_3 of the first sensor fixing module 10a and a fourth horizontal part 11b_4 of the second sensor fixing module 10b, and a fourth elastic part 21_4 may be connected to a fourth horizontal part 11a_4 of the first sensor fixing module 10a and a first horizontal part 11b_1 of the second sensor fixing module 10b.

That is, when the horizontal part 11 equipped with the dial 13 among the horizontal parts 11 is a first horizontal part 11_1, an n^{th} (n is a natural number) elastic part 21_n may be connected to an n^{th} horizontal part 11_n of the first sensor fixing module 10a and an (n+1)^{th} horizontal part 11_n+1 of the second sensor fixing module 10b. The elastic part 21 may be disposed diagonally with respect to a height direction of the groundwater monitoring sensor. The elastic part 21 may be diagonally disposed so that the expansion module 20 may buffer an external force acting on the groundwater monitoring sensor in a wide region.

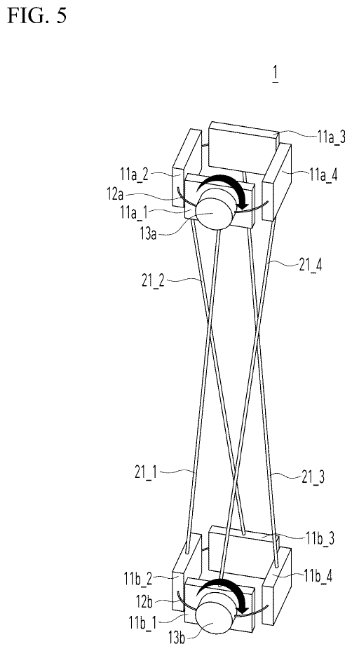

FIG. 5 is a view illustrating an embodiment in which an interval between the horizontal parts 11 is adjusted by rotating the dial 13 according to one embodiment.

Referring to FIG. 5, when the dial 13 of each sensor fixing module 10 rotates in the first direction, the interval between the horizontal parts 11 may decrease, and an area of a region surrounded by the horizontal parts 11 may also decrease. That is, in order to be installed on a groundwater monitoring sensor with a small diameter, the dial 13 may be rotated in the first direction so that the region surrounded by the horizontal parts 11 may be adjusted to fit a diameter of the groundwater monitoring sensor.

Specifically, the wire 12 connecting the horizontal parts 11 may be wound when the dial 13 is rotated in the first direction. When the wire 12 is wound on the dial 13, the interval between the horizontal parts 11 may decrease, and the sensor guard 1 may accommodate a groundwater monitoring sensor with a smaller diameter.

In this case, the dial 13 may be locked not to rotate in the second direction which is the direction opposite to the first direction. That is, the dial 13 may be fixed such that the wire 12 is not unwound after the wire 12 is wound, and accordingly, the interval between the horizontal parts 11 may be decreased to fit a diameter of the groundwater monitoring sensor and then fixed not to be increased.

The dial 13 may include a ratchet gear such that the dial 13 may be rotated only in the first direction and locked not to rotate in the second direction. The ratchet gear may include a ratchet wheel and a pawl, and the ratchet wheel may be a wheel with asymmetrical teeth. In general, surface of the ratchet wheel is generally inclined to facilitate rotation, and an opposite surface of the ratchet wheel is nearly vertical to prevent rotation. The pawl may be a lever that is continuously applies pressure to the teeth of the ratchet wheel through a spring. The pawl, together with the ratchet wheel, may allow the dial 13 to rotate only in the first direction by allowing rotation in a specific direction while blocking rotation in the opposite direction.

Specifically, when the dial 13 is turned in the first direction, an inclined surface of the ratchet wheel comes into contact with the pawl to allow the pawl to slide between the teeth. In such a process, the pawl may move to the next tooth to allow the ratchet wheel to freely rotate. On the other hand, when the dial 13 is to rotate in the second direction, the surface of the ratchet wheel that is nearly vertical meets the pawl. In this case, the pawl is caught on the tooth to physically prevent the ratchet wheel from rotating in the second direction.

To this end, the pawl continuously applies pressure to the teeth of the ratchet wheel through a spring and thus is firmly fixed to the teeth when rotating in the second direction. Thus, the rotation of the dial 13 in the second direction is securely blocked, thereby preventing the horizontal parts 11 from being separated from the groundwater monitoring sensor.

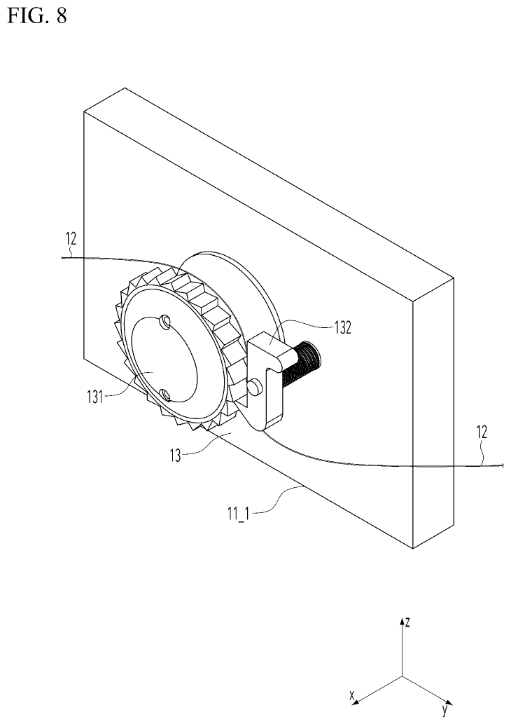

According to one embodiment, the ratchet wheel and the pawl of the dial 13 are uncoupled from each other, thereby allowing the rotation of the dial 13 in the second direction. An embodiment in which the rotation of the dial 13 in the second direction is allowed will be described below with reference to FIG. 8.

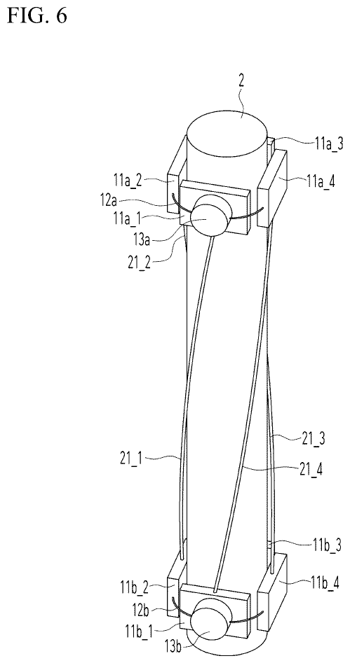

FIG. 6 is a view illustrating a state in which the sensor fixing module 10 is in close contact with a groundwater monitoring sensor 2 according to one embodiment.

Referring to FIG. 6, as the dial 13 rotates in the first direction, the sensor fixing module 10 may come into close contact with the groundwater monitoring sensor 2. Before the sensor guard 1 expands in an outward direction of a sensor, the sensor guard 1 may not be in full contact with the groundwater monitoring sensor 2 and may be fastened to the groundwater monitoring sensor 2 by leaving a minimum interval that allows the sensor fixing module 10 to move in a height direction of the sensor.

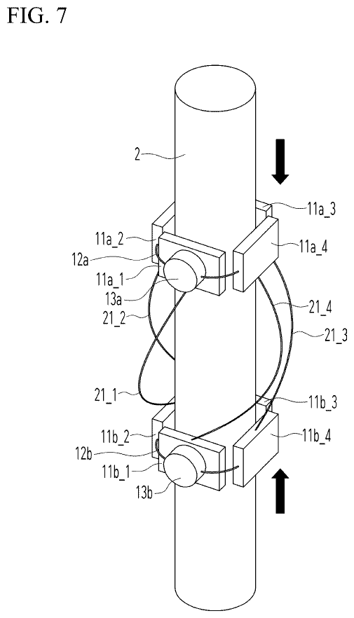

FIG. 7 is a view illustrating an embodiment in which the expansion module 20 is expanded by operating the sensor fixing module 10 according to one embodiment.

Referring to FIG. 7, the first sensor fixing module 10a may be moved downward, and the second sensor fixing module 10b may be moved upward so that an interval between the first sensor fixing module 10a and the second sensor fixing module 10b may be decreased. Thus, the elastic part 21 of the expansion module 20 may be bent in an outward direction of the groundwater monitoring sensor 2.

When an external impact is applied to a product, the elastic part 21 may be bent in an inward direction of the groundwater monitoring sensor 2 to absorb impact energy. When an external force is applied to a plurality of elastic parts 21, the impact may be distributed to each elastic part 21, and when the elastic part 21 is bent in the inward direction of the groundwater monitoring sensor 2 to an elastic limit of the elastic part 21, the elastic part 21 may reflect the impact, thereby preventing the impact from being applied to the groundwater monitoring sensor 2.

After the elastic part 21 is bent outward to cause the expansion module 20 to have an expanded shape, the dial 13 of each sensor fixing module 10 may be rotated in the first direction so that the sensor fixing module 10 may be in close contact with and fixed to the groundwater monitoring sensor 2. When the sensor fixing module 10 is in close contact with and fixed to the groundwater monitoring sensor 2, the sensor fixing module 10 may not move in a vertical direction and an outer circumferential direction of the cylindrical sensor.

Thereafter, in order to disassemble the sensor guard 1 from the groundwater monitoring sensor 2, a position of the ratchet wheel or pawl of the dial 13 may be adjusted to rotate the dial 13 in the second direction. When the dial 13 is rotated in the second direction, the interval between the horizontal parts 11 be increased, the close contact fixation of the sensor fixing module 10 may be released, thereby separating the sensor guard 1 from the groundwater monitoring sensor 2.

Although an embodiment in which, in the sensor guard 1 according to the embodiment of the present disclosure, one elastic part 21 is connected to each horizontal part 11 of the sensor fixing module 10 has been described, in the sensor guard 1 according to the embodiment of the present disclosure, the plurality of elastic parts 21 may be connected to each horizontal part 11, and some of the elastic parts 21 may be disposed outside the groundwater monitoring sensor 2 to be misaligned with each other. The elastic parts 21 disposed to be misaligned with each other may absorb an external impact more efficiently.

FIG. 8 is a view illustrating the dial 13 according to one embodiment.

Referring to FIG. 8, the dial 13 may include a ratchet wheel 131 and a pawl 132, and when the ratchet wheel 131 is rotated in the first direction, the wire 12 may be wound on a winding part disposed at a lower portion (−x direction) of the ratchet wheel 131. A spring may be connected to a lower portion of the pawl 132 to apply torque such that the pawl 132 may be in continuous contact with teeth of the ratchet wheel 131.

In the embodiment of FIG. 8, the ratchet wheel 131 may continuously rotate counterclockwise on a y-z plane and may be prevented from rotating clockwise on the y-z plane by a tooth surface that is nearly vertical. While the ratchet wheel 131 rotates counterclockwise, for each tooth, the pawl 132 may rotate a predetermined angle in the clockwise direction and then return in the counterclockwise direction by a restoring force of a spring.

When the ratchet wheel 131 and the pawl 132 are positioned at the same level in an x-axis direction, the ratchet wheel 131 and the pawl 132 may engage with each other so that only rotation in the first direction may be allowed. According to an embodiment of the present disclosure, a level of any one of the ratchet wheel 131 and the pawl 132 may be adjusted in the x-axis direction, and when the level is adjusted, the ratchet wheel 131 and the pawl 132 do not engage with each other, thereby allowing rotation in the second direction. For example, the ratchet wheel 131 may be extended in a +x direction, and the pawl 132 may be positioned in the −x direction relative to the ratchet wheel 131 so that the ratchet wheel 131 and the pawl 132 may not engage with each other. Accordingly, the wire 12 that has been wound on the winding part may be unwound again, and the interval between the horizontal parts 11 may be increased again.

Afterwards, the ratchet wheel 131 or pawl 132 of which the level has been adjusted to tighten the sensor guard 1 to fit the groundwater monitoring sensor 2 may be adjusted again to the original position so that the ratchet wheel 131 and the pawl 132 may engage with each other, and the dial 13 may be rotated only in the first direction to adjust the sensor fixing module 10 to fit the groundwater monitoring sensor 2.

Although an embodiment in which, in the dial 13 according to the embodiment of the present disclosure, the pawl 132 is positioned on one side surface of the ratchet wheel 131 to allow rotation in only one direction, a positional relationship between the pawl 132 and the ratchet wheel 131 is not limited thereto. For example, the pawl 132 may be provided to surround an outer circumferential surface of the ratchet wheel 131, or the pawl 132 may be positioned at a central portion while the ratchet wheel 131 surrounds an outer circumferential surface of the pawl 132.

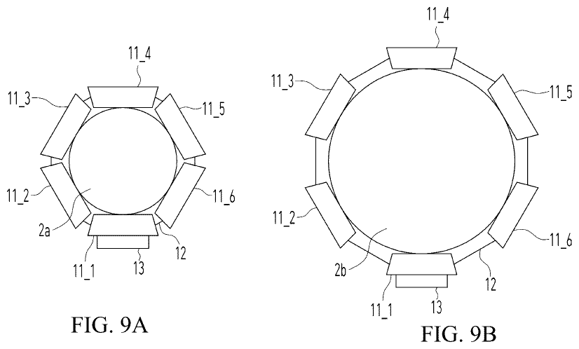

FIG. 9 shows views illustrating an embodiment in which the sensor guard 1 is installed on each of groundwater monitoring sensors with a plurality of sizes according to one embodiment.

In the above embodiment, an embodiment in which four horizontal parts 11 are connected through the wire 12 and the sensor guard 1 is installed on each of the groundwater monitoring sensors with a plurality of sizes has been described, but the number of the horizontal parts 11 of the present disclosure may not be limited thereto.

Referring to FIG. 9, the number of horizontal parts 11_1 to 11_6 may be 6, and when the wire 12 is most tightly wound on the dial 13, a shape of the sensor fixing module 10 viewed from above may be close to a regular hexagon.

A diameter of the first groundwater monitoring sensor 2a of FIG. 9A may be smaller than a diameter of a second groundwater monitoring sensor 2b of FIG. 9B. The dial 13 of the sensor guard 1 may wind more wire 12 when coupled to the first groundwater monitoring sensor 2a to maintain an interval between the horizontal parts 11 narrow, and as compared to when coupled to the first groundwater monitoring sensor 2a, the dial 13 of the sensor guard 1 may wind less wire 12 when coupled to the second groundwater monitoring sensor 2b to maintain an interval between the horizontal parts 11 wider.

Through a sensor guard according to embodiments of the present disclosure, a groundwater monitoring sensor can be installed through a groundwater well without damage. In particular, a sensor guard according to embodiments of the present disclosure can be easy to operate, can be installed on sensors with various sizes, and can be inserted into a groundwater well with a narrow size.

Effects that can be obtained from the exemplary embodiments of the present disclosure are not limited to the effects described above, and other effects that are not described can be clearly derived and understood by a person having ordinary skill in the art to which the exemplary embodiments of the present disclosure belong from the following description. That is, unintended effects resulting from the implementation of the exemplary embodiments of the present disclosure can also be derived from the exemplary embodiments of the present disclosure by a person having ordinary skill in the art.

What is claimed is:

1. A sensor guard installable on various types of groundwater monitoring sensors inserted into a groundwater well, the sensor guard comprising:
at least one sensor fixing module fixed to the groundwater monitoring sensor in one direction of the groundwater monitoring sensor; and
an expansion module connected to the sensor fixing module to expand in an outward direction of the groundwater monitoring sensor according to an operation of the sensor fixing module,
wherein an external force applied to the sensor guard is buffered by the expansion module.

2. The sensor guard of claim 1, wherein the sensor fixing module is provided as a plurality of sensor fixing modules, and
the expansion module is connected between the plurality of sensor fixing modules.

3. The sensor guard of claim 2, wherein each of the sensor fixing modules includes a plurality of horizontal parts surrounding the groundwater monitoring sensor, and
the horizontal parts are connected through a wire by which an interval between the horizontal parts is adjusted.

4. The sensor guard of claim 3, wherein, in the expansion module, an elastic part bendable in an inward or outward direction of the groundwater monitoring sensor is connected to each of the horizontal parts, and
a degree of expansion of the elastic part outward from the groundwater monitoring sensor is determined according to an interval between the plurality of sensor fixing modules.

5. The sensor guard of claim 3, wherein each of the sensor fixing modules includes a dial on which the wire is wound, and
when the dial rotates in a first direction, the wire is wound on the dial so that the interval between the horizontal parts decreases.

6. The sensor guard of claim 5, wherein the dial is locked not to rotate in a second direction opposite to the first direction, and
when the dial is pushed toward the groundwater monitoring sensor, the dial is unlocked to allow rotation in the second direction.

7. The sensor guard of claim 6, wherein, when the dial rotates in the second direction, the interval between the horizontal parts increases.
